(19) United States
(12) Patent Application Publication
(10) Pub. No.: US 2010/0298564 A1
(43) Pub. Date: Nov. 25, 2010

(54) PREPARATION OF AMORPHOUS VALGANCICLOVIR HYDROCHLORIDE

(76) Inventors: Venu Nalivela, Hyderabad (IN); Arjun Kumar Tummala, Visakhapatnam (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/785,558

(22) Filed: May 24, 2010

Related U.S. Application Data
(60) Provisional application No. 61/291,133, filed on Dec. 30, 2009.

(30) Foreign Application Priority Data
May 25, 2009 (IN) 1206/CHE/2009

(51) Int. Cl. C07D 473/18

(52) U.S. Cl. 544/276

(57) ABSTRACT

The present application relates to processes for the preparation amorphous valganciclovir hydrochloride, comprising combining a solution of valganciclovir with an anti-solvent.

### INTRODUCTION

An aspect of the present invention relates to processes for preparation of amorphous valganciclovir hydrochloride.

Valganciclovir hydrochloride has a chemical name L-valine, 2-[(2-amino-1,6-dihydro-6-oxo-9H-purin-9-yl)-methoxy]-3-hydroxypropanyl ester, monohydrochloride and is represented by structural Formula I.

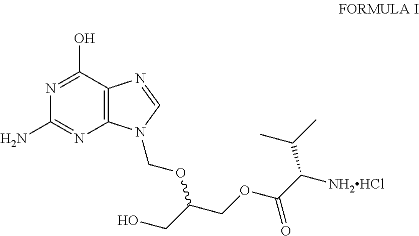

Valganciclovir hydrochloride is an active ingredient in products prescribed for the treatment of cytomegalovirus (CMV) retinitis in patients with acquired immunodeficiency syndrome (AIDS), and for the prevention of cytomegalovirus (CMV) disease in kidney, heart, and kidney-pancreas transplant patients at high risk (Donor CMV seropositive/recipient CMV seronegative [(D+/R−)]).

Indian Patent Application No. 1697/DEL/2005 discloses a process for preparation of amorphous valganciclovir hydrochloride, comprising spray drying a solution of valganciclovir hydrochloride in a spray drying system fitted with a rotary atomizer.

International Patent Application No. WO 2005/021549 A1 discloses an amorphous form of valganciclovir hydrochloride and a process for its preparation involving spray drying or vacuum distillation techniques.

However, spray drying is not a preferred technique, especially for manufacturing on a commercial scale.

International Patent Application No. WO 2005/021549 A1 also discloses a process for the preparation of amorphous valganciclovir hydrochloride, comprising:
a) dissolving crystalline valganciclovir hydrochloride in water;
b) adding an organic solvent capable of forming an azeotropic mixture with water;
c) azeotropically removing water from the mixture of step b);
d) treating the mixture obtained in step c) with a further organic solvent; and
e) isolating amorphous valganciclovir hydrochloride from the mass.

The above process results in high levels of residual solvent/organic volatile impurities that are not in accordance with the limits as required by ICH (International Conference on Harmonization of Technical Requirements for Registration of Pharmaceuticals for Human Use) guidelines, thus making the product non-complaint for sales in regulated markets. Hence, the prior process is not suitable for commercial production of amorphous valganciclovir hydrochloride.

There remains a need for a simple, cost effective and industrially viable process for the production of amorphous valganciclovir hydrochloride, which meets the acceptable levels of residual solvent/organic volatile impurities contents in accordance with limits as required by ICH guidelines.

### SUMMARY

In an aspect, the present application provides processes for the preparation of amorphous valganciclovir hydrochloride, an embodiment including:

a) providing a solution of valganciclovir hydrochloride in a suitable solvent;

b) combining the solution of valganciclovir hydrochloride obtained in step a) with an anti-solvent;

c) isolating the solid from the mixture of step b) and optionally washing with an anti-solvent; and

d) optionally, drying the solid to obtain amorphous valganciclovir hydrochloride.

In embodiments, processes for the preparation of amorphous valganciclovir hydrochloride include:

a) providing a solution of valganciclovir hydrochloride in a solvent;

b) combining the solution of valganciclovir hydrochloride with an anti-solvent;

c) isolating the solid from the mixture of step b) and optionally delumping or milling the resulting solid; and

d) optionally, drying the solid to obtain amorphous valganciclovir hydrochloride.

### BRIEF DESCRIPTION OF THE DRAWING

FIG. 1 is an illustration of a powder X-ray diffraction (“PXRD”) pattern of amorphous valganciclovir hydrochloride prepared according to Examples 1-5.

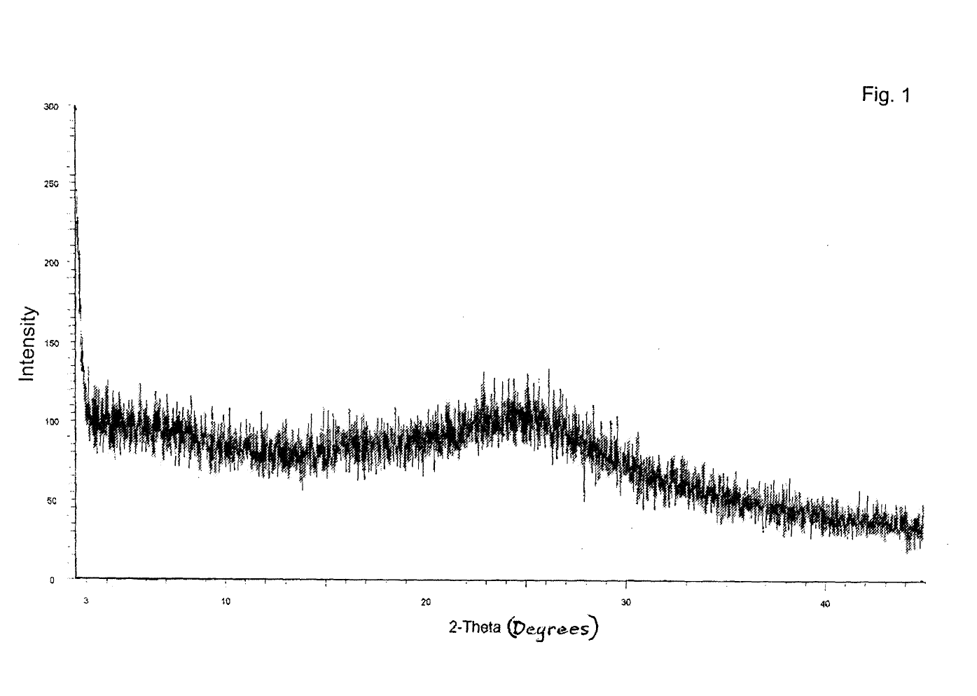

### DETAILED DESCRIPTION

As set forth herein, the present application provides processes for the preparation of amorphous valganciclovir hydrochloride, embodiments including:

a) providing a solution of valganciclovir hydrochloride in a suitable solvent;

b) combining the solution of valganciclovir hydrochloride of step a) with an anti-solvent;

c) isolating the solid from the mixture of step b) and optionally washing with an anti-solvent; and

d) optionally, drying the solid to obtain amorphous valganciclovir hydrochloride.

Step a) involves providing a solution of valganciclovir hydrochloride in a suitable solvent.

The solution of valganciclovir hydrochloride may be obtained by dissolving valganciclovir hydrochloride in a suitable solvent, or it may be obtained directly from a reaction mixture containing the compound from a valganciclovir synthesis.

Suitable solvents that may be employed for providing a solution of valganciclovir hydrochloride in step a) include solvents in which valganciclovir hydrochloride is freely soluble, such as, but not limited to: alcohols including methanol, ethanol, and methoxyethanol; a glycol such as ethylene glycol; N,N-dimethylformamide; dimethylsulfoxide; N,N-dimethylacetamide; water; and any mixtures thereof.

The temperatures at which a solution may be obtained in step a) range from about 0° C. to about the reflux temperature of the solvent that is used.

Step b) involves combining the solution of valganciclovir hydrochloride obtained in step a) with an anti-solvent.

An anti-solvent, as used herein, refers to a liquid in which valganciclovir hydrochloride is poorly soluble. Some anti-solvents that may be used in step b) include, but are not limited to: isopropyl alcohol, 1-propanol, 1-butanol, 2-ethoxymethanol, methyl acetate, ethyl acetate, isopropyl acetate, acetone, methyl ethyl ketone, diethyl ether, methyl t-butyl ether, tetrahydrofuran, toluene, acetonitrile, and any mixtures thereof.

Temperatures for combining a solution of valganciclovir hydrochloride with an anti-solvent in step b) range from about −20° C. to about 50° C., or about −15° C. to about 20° C., or about −10° C. to about 10° C., or about 0° C. to about 5° C.

Times for combining a solution of valganciclovir hydrochloride with an anti-solvent in step (b) generally are less than about 90 minutes, or less than about 60 minutes, or less than about 40 minutes, or any other suitable times.

Combining can involve adding a solution containing valganciclovir hydrochloride to an anti-solvent, or adding an anti-solvent to a solution comprising valganciclovir hydrochloride.

Step c) involves isolation of a solid from the mixture of step b) and optionally washing the obtained solid with an anti-solvent.

The solid produced in step b) may be isolated by, for example, filtration through a paper, cloth, polymeric membrane, etc., using a Nutsche filter, decantation, centrifugation, gravity filtration, or suction filtration. The isolation may be carried out in the presence or absence of an inert atmosphere such as nitrogen, argon, neon, or helium. The isolation may be carried out at atmospheric pressure, under positive pressure, or under reduced pressure.

The isolated solid is optionally washed with an anti-solvent to remove occluded mother liquor. The anti-solvents that may be used in step c) include, but are not limited to: isopropyl alcohol, 1-propanol, 1-butanol, 2-ethoxymethanol, methyl acetate, ethyl acetate, isopropyl acetate, acetone, methyl ethyl ketone, diethyl ether, methyl t-butyl ether, tetrahydrofuran, toluene, acetonitrile, and any mixtures thereof.

Washing may be carried out by pouring the anti-solvent onto a bed of solid, or by mixing, stirring, or shaking the obtained solid together with the anti-solvent.

Step d) involves optional drying of the solid obtained in step c).

Drying may be suitably carried out using a tray dryer, vacuum oven, air oven, fluidized bed dryer, spin flash dryer, flash dryer, and the like, at atmospheric pressure or under reduced pressure. Drying may be carried out at temperatures less than about 120° C., or less than about 100° C., or less than about 80° C., or any other suitable temperatures, in the presence or absence of an inert atmosphere such as nitrogen, argon, neon, or helium. The drying may be carried out for any desired time periods to achieve the desired quality of the product, such as, for example, about 1 to about 24 hours, or longer.

In embodiments, the present application provides processes for the preparation of amorphous valganciclovir hydrochloride, including:

a) providing a solution of valganciclovir hydrochloride in a solvent;

b) combining the solution of valganciclovir hydrochloride with an anti-solvent;

c) isolating a solid from the mixture of step b) and delumping or milling the resulting solid; and

d) optionally, drying the solid.

Step a) involves providing a solution of valganciclovir hydrochloride in a suitable solvent.

The solution of valganciclovir hydrochloride may be obtained by dissolving valganciclovir hydrochloride in a suitable solvent, or it may be obtained directly from a reaction mixture resulting from synthesis of valganciclovir.

Suitable solvents that may be used for providing a solution of valganciclovir hydrochloride in step a) include solvents in which valganciclovir hydrochloride is freely soluble, such as, but not limited to: alcohols such as methanol, ethanol, methoxyethanol; a glycol such as ethylene glycol; N,N-dimethylformamide; dimethylsulfoxide; N,N-dimethylacetamide; water; and any mixtures thereof.

The temperatures at which a solution may be obtained in step a) range from about 0° C. to about the reflux temperature of the solvent that is used.

Step b) involves combining the solution of valganciclovir hydrochloride with an anti-solvent.

An anti-solvent is a liquid in which valganciclovir hydrochloride is poorly soluble. Some anti-solvents that may be used in step b) include, but are not limited to, isopropyl alcohol, 1-propanol, 1-butanol, 2-ethoxymethanol, methyl acetate, ethyl acetate, isopropyl acetate, acetone, methyl ethyl ketone, diethyl ether, methyl t-butyl ether, tetrahydrofuran, toluene, acetonitrile, and mixtures thereof.

Temperatures for combining a solution of valganciclovir hydrochloride with an anti-solvent in step b) range from about −20° C. to about 50° C., or about −15° C. to about 20° C., or about −10° C. to about 10° C., or about 0° C. to about 5° C.

Times for combining a solution of valganciclovir hydrochloride with an anti-solvent in step b) range from about 10 minutes to about 90 minutes, or about 15 minutes to about 60 minutes, or about 20 minutes to about 40 minutes.

Step c) involves isolation of a solid from the mixture of step b) and delumping or milling the obtained solid.

The solid obtained from step b) may be isolated by, for example, filtration through a cloth, paper, or polymeric membrane, using a Nutsche filter, decantation, centrifugation, gravity filtration or suction filtration. The isolation may be carried out in the presence or absence of an inert atmosphere such as nitrogen, argon, neon, or helium.

The solid recovered after isolation may be subjected to delumping or milling to obtain a solid having desired particle sizes.

Delumping or milling may be carried out using equipment such as ball, roller, and hammer mills, jet mills, co-mills, and multi-mills.

Step d) involves drying the solid obtained in step c).

Drying may be suitably carried out using a tray dryer, vacuum oven, air oven, fluidized bed dryer, spin flash dryer, flash dryer, and the like, at atmospheric pressure or under reduced pressure. Drying may be carried out at temperatures less than about 120° C., or less than about 100° C., or less than about 80° C., or any other suitable temperatures, in the presence or absence of an inert atmosphere such as nitrogen, argon, neon, or helium. The drying may be carried out for any desired time periods to achieve the desired quality of the product, such as, for example, about 1 to about 24 hours, or longer.

Certain specific aspects and embodiments of the present application are explained in more detail with reference to the following examples, which are provided only for purposes of illustration and should not be construed as limiting the scope of the application in any manner.

### Example 1

Methyl ethyl ketone (1050 mL) is charged into a cylindrical flask, then cooled and stirred at 1.5° C. for 60 minutes. Valganciclovir hydrochloride (15 g) is dissolved in methanol (105 mL) at 28° C., stirred for 15 minutes, and filtered. The filtrate is slowly added to the cooled methyl ethyl ketone at 1.5° C. over 40 minutes, followed by stirring the mixture for 65 minutes. The mass is divided into 4 parts, which are treated individually.

Part A: 300 mL of the mass is filtered through a pressure Nutsche filter in an inert atmosphere and washed with ethyl acetate (100 mL). The wet solid is dried at 80° C. for 23 hours, to obtain 3.11 g of amorphous valganciclovir hydrochloride. Solvent content: methanol (not detected); methyl ethyl ketone (1242.5 ppm); ethyl acetate (1241 ppm).

Part B: 300 mL of the mass is filtered through a pressure Nutsche filter in an inert atmosphere and washed with chilled acetone (100 mL). The wet solid is dried at 80° C. for 23 hours, to obtain 3.29 g of amorphous valganciclovir hydrochloride. Solvent content: methanol (not detected); methyl ethyl ketone (1385.9 ppm); acetone (not detected).

Part C: 300 mL of the mass is filtered through a pressure Nutsche filter in an inert atmosphere and washed with acetonitrile (100 mL). The wet solid is dried at 80° C. for 23 hours, to obtain 3.11 g of amorphous valganciclovir hydrochloride. Solvent content: methanol (not detected); methyl ethyl ketone (1385.9 ppm); acetonitrile (not detected).

Part D: 300 mL of the mass is filtered through a pressure Nutsche filter in an inert atmosphere and washed with isopropanol (100 mL). The wet solid is dried at 80° C. for 23 hours, to obtain 3.21 g of amorphous valganciclovir hydrochloride. Solvent content: methanol (not detected); methyl ethyl ketone (1602.3 ppm); isopropanol (not detected).

### Example 2

Methyl ethyl ketone (700 mL) is charged into a cylindrical flask, then cooled and stirred at 1.5° C. for 60 minutes. Valganciclovir hydrochloride (10 g) is dissolved in methanol (70 mL) at 28° C., stirred for 15 minutes and filtered. The filtrate is slowly added to the cooled methyl ethyl ketone at 1.5° C. over 20 minutes, followed by stirring the mixture for 70 minutes. The formed solid is filtered through a pressure Nutsche filter in an inert atmosphere and washed with chilled acetone (200 mL). The obtained solid is divided two parts, which are treated individually.

Part A: 4.017 g of the solid is dried under atmospheric pressure at 80° C. for 24 hours to obtain 3.91 g of amorphous valganciclovir hydrochloride. Solvent content: methanol (not detected); methyl ethyl ketone (2742 ppm); acetone (not detected).

Part B: 3.97 g of the solid is dried under reduced pressure at 80° C. for 23 hours to obtain 3.92 g of amorphous valganciclovir hydrochloride. Solvent content: methanol (not detected); methyl ethyl ketone (3176 ppm); acetone (not detected).

### Example 3

Methyl ethyl ketone (700 mL) is charged into a cylindrical flask, then cooled and stirred at 0° C. for 40 minutes. Valganciclovir hydrochloride (10 g) is dissolved in methanol (70 mL) at 29° C., stirred for 15 minutes and filtered. The filtrate is slowly added to the cooled methyl ethyl ketone at 0° C. over 40 minutes, followed by stirring the mixture for 70 minutes. The formed solid is filtered through a pressure Nutsche filter in an inert atmosphere and washed with isopropanol (2×100 mL). The solid is dried under reduced pressure at 80° C. for 22 hours to obtain 8.83 g of amorphous valganciclovir hydrochloride. Solvent content: methanol (19.68 ppm); methyl ethyl ketone (1836.76 ppm); isopropanol (1089.66 ppm).

### Example 4

Methyl ethyl ketone (700 mL) is charged into a cylindrical flask, then cooled and stirred at 0° C. for 40 minutes. Valganciclovir hydrochloride (10 g) is dissolved in methanol (70 mL) at 28° C., stirred for 20 minutes and filtered. The filtrate is slowly added to the cooled methyl ethyl ketone at 0° C. over 30 minutes, followed by stirring the mixture for 95 minutes. The mass is filtered through a pressure Nutsche filter in an inert atmosphere. The wet solid is subjected to delumping in a co-mill for 15 minutes. The obtained solid is dried under reduced pressure at 80° C. for about 23 hours to obtain 6.45 g of amorphous valganciclovir hydrochloride. Solvent content: methanol (44.8 ppm); methyl ethyl ketone (2021.5 ppm).

### Example 5

Isopropyl alcohol (1000 mL) is charged into a cylindrical flask, then cooled and stirred at 0° C. for 40 minutes. Valganciclovir hydrochloride (10 g) is dissolved in methanol (70 mL) at 28° C., stirred for 15 minutes and filtered. The filtrate is slowly added to the cooled isopropanol at 0° C. over 50 minutes, followed by stirring the mixture for 80 minutes. The formed solid is filtered through a pressure Nutsche filter in an inert atmosphere. The wet solid is divided into two parts, which are individually treated.

Part A: 4.78 g of the solid is dried under reduced pressure at 80° C. for 24 hours to obtain 4.35 g of amorphous valganciclovir hydrochloride. Solvent content: methanol (not detected); isopropanol (2933.66 ppm).

Part B: 2.65 g of the solid is dried under atmospheric pressure at 80° C. for 21 hours to obtain 2.38 g of amorphous valganciclovir hydrochloride. Solvent content: methanol (not detected); isopropanol (1045.34 ppm).

1. A process for preparing amorphous valganciclovir hydrochloride, comprising:
a) providing a solution of valganciclovir hydrochloride in a solvent;
b) combining the solution of valganciclovir hydrochloride with an anti-solvent; and
c) isolating a solid from the mixture of b).

2. The process of claim 1, further comprising washing the solid with an anti-solvent.

3. The process of claim 1, further comprising delumping or milling the solid.

4. The process of claim 1, further comprising drying the solid at temperatures less than about 120° C.

5. The process of claim 1, wherein a solvent comprises an alcohol, a glycol, N,N-dimethylformamide, dimethylsulfoxide, N,N-dimethylacetamide, water, or any mixtures thereof.

6. The process of claim 1, wherein a solvent comprises methanol.

7. The process of claim 1, wherein an anti-solvent comprises isopropyl alcohol, 1-propanol, 1-butanol, 2-ethoxymethanol, methyl acetate, ethyl acetate, isopropyl acetate, acetone, methyl ethyl ketone, diethyl ether, methyl t-butyl ether, tetrahydrofuran, toluene, acetonitrile, or any mixtures thereof.

8. The process of claim 1, wherein a solution of valganciclovir hydrochloride is combined with an anti-solvent at about −20° C. to about 50° C.

9. The process of claim 1, wherein combining comprises adding a solution of valganciclovir hydrochloride to an anti-solvent.

10. The process of claim 1, wherein times for combining a solution of valganciclovir hydrochloride with an anti-solvent range from about 10 minutes to about 90 minutes.

11. The process of claim 2, wherein a solid is washed with an anti-solvent comprising isopropyl alcohol, 1-propanol, 1-butanol, 2-ethoxymethanol, methyl acetate, ethyl acetate, isopropyl acetate, acetone, methyl ethyl ketone, diethyl ether, methyl t-butyl ether, tetrahydrofuran, toluene, acetonitrile, or any mixtures thereof.

12. A process for preparing amorphous valganciclovir hydrochloride, comprising:
a) providing a solution of valganciclovir hydrochloride in a solvent comprising an alcohol;
b) combining the solution of valganciclovir hydrochloride with an anti-solvent; and
c) isolating a solid from the mixture of b).

13. The process of claim 12, wherein a solvent comprises methanol.

14. The process of claim 12, wherein an anti-solvent comprises isopropyl alcohol, 1-propanol, 1-butanol, 2-ethoxymethanol, methyl acetate, ethyl acetate, isopropyl acetate, acetone, methyl ethyl ketone, diethyl ether, methyl t-butyl ether, tetrahydrofuran, toluene, acetonitrile, or any mixtures thereof.

15. The process of claim 12, wherein an anti-solvent comprises methyl ethyl ketone or isopropyl alcohol.

16. The process of claim 12, wherein a solution of valganciclovir hydrochloride is combined with an anti-solvent at about −20° C. to about 50° C.

17. The process of claim 12, wherein combining comprises adding a solution of valganciclovir hydrochloride to an anti-solvent.

18. The process of claim 12, wherein times for combining a solution of valganciclovir hydrochloride with an anti-solvent range from about 10 minutes to about 90 minutes.

19. A process for preparing amorphous valganciclovir hydrochloride, comprising:
a) providing a solution of valganciclovir hydrochloride in a solvent comprising methanol;
b) combining the solution of valganciclovir hydrochloride with an anti-solvent comprising methyl ethyl ketone or isopropanol; and
c) isolating a solid from the mixture of b).
